(19) United States
(12) Patent Application Publication
(10) Pub. No.: US 2018/0214211 A1
(43) Pub. Date: Aug. 2, 2018

(54) SIDE-FIRING LASER FIBER WITH PROTECTIVE TIP AND RELATED METHODS

(71) Applicant: Boston Scientific Scimed, Inc., Maple Grove, MN (US)

(72) Inventors: Brian HANLEY, Framingham, MA (US); Jessica HIXON, Miami, FL (US); Christopher L. OSKIN, Grafton, MA (US); Edward SINOFSKY, Dennis, MA (US)

(73) Assignee: Boston Scientific Scimed, Inc., Maple Grove, MN (US)

(21) Appl. No.: 15/939,506

(22) Filed: Mar. 29, 2018

Related U.S. Application Data
(63) Continuation of application No. 12/467,730, filed on May 18, 2009, now abandoned.
(60) Provisional application No. 61/054,280, filed on May 19, 2008.

(51) Int. Cl. A61B 18/24; A61B 18/22

(52) U.S. Cl. CPC A61B 18/24; A61B 2018/2272; Y10T 29/49982

(57) ABSTRACT

A method and an apparatus according to an embodiment of the invention includes a reflector disposed within a capillary for use in side-firing optical fibers. An outer member or cap can be used to protect the capillary when being inserted through a catheter or endoscope. The endoscope is then at least partially inserted into a patient's body to provide laser-based medical treatment. In some embodiments, a multilayer dielectric coating can be disposed on an angled surface of the reflector. In other embodiments, a multilayer dielectric coating can be positioned between a distal end surface of the reflector and an inner side of a distal end portion of the capillary. The coated reflector can be configured to increase laser energy redirected from a first portion of an optical path to a second portion of the optical path that is non-parallel to the first portion.

### RELATED APPLICATION

This application claims priority to and the benefit of U.S. Provisional Application No. 61/054,280, filed on May 19, 2008, entitled “Side-Firing Laser with Protective Tip and Related Methods,” which is incorporated herein by reference in its entirety.

### BACKGROUND

The invention relates generally to medical devices and more particularly to side-firing optical fibers and methods for using such devices.

Laser-based surgical procedures using side-firing optical fibers can provide a medical practitioner with more control when applying laser energy to the appropriate treatment area. Passing the distal end portion of the optical fiber through an endoscope during surgery, however, may damage, scratch, degrade, and/or deform the distal end portion of the optical fiber. To protect the optical-fiber end portion, a capillary and/or a metal cap or cannula, usually made of surgical grade stainless steel, can be placed over the optical-fiber end portion. Once the optical-fiber end portion is properly positioned for treatment, the laser energy can be applied to the target area.

During use of the device, a portion of the laser energy can leak from the optical-fiber end, reducing the efficiency with which laser energy is delivered to the treatment area and/or increasing overheating of the metal cap that is typically used to protect the optical fiber. Cooling of the device may be needed to operate at a safe temperature. In some instances, the overheating that can occur from the laser energy leakage can affect the mechanical and/or optical properties of the optical-fiber end portion, the capillary and/or the metal cap. In other instances, the overheating that can occur from the laser energy leakage can be sufficiently severe to damage the optical-fiber end portion, the capillary and/or the metal cap.

Overheating can also occur from the use of reflectors such as metallic reflectors or tips configured to redirect or bend an optical beam about 90 degrees from its original propagation path based on total internal reflection (TIR). Because metallic reflectors do not reflect 100% of the optical beam, the energy associated with the non-reflected portion of the optical beam can be absorbed by the metallic reflector and the metallic reflector can self heat. For TIR-based tips, a portion of the optical beam can leak through and heat up a protective metal cap positioned on a distal end of the tip. Furthermore, the glass capillary tubing that is generally used on the TIR-based tips can become damaged as tissue is ablated and impacts against the glass capillary tubing.

Thus, a need exists for optical-fiber end portions that can increase side-fired laser energy, increase device longevity, increase transmission efficiency, reduce overheating, and/or increase patient safety.

### SUMMARY

An apparatus includes a reflector disposed within a capillary for use in side-firing optical fibers. An outer member or cap can be used to protect the capillary when being inserted through a catheter or endoscope. The endoscope is then at least partially inserted into a patient's body to provide laser-based medical treatment. In some embodiments, a multilayer dielectric coating can be disposed on an angled surface of the reflector. In other embodiments, a multilayer dielectric coating can be positioned between a distal end surface of the reflector and an inner side of a distal end portion of the capillary. The coated reflector can be configured to increase the laser energy redirected from a first portion of an optical path to a second portion of the optical path that is non-parallel to the first portion.

### BRIEF DESCRIPTION OF THE DRAWINGS

FIG. 1 is a schematic representation of a side-firing optical fiber system according to an embodiment of the invention.

FIG. 2 is a cross-sectional view of an optical-fiber distal end portion according to an embodiment of the invention.

FIG. 3A is cross-sectional view of a side-firing optical fiber with capillary and reflective member, according to an embodiment of the invention.

FIG. 3B is cross-sectional view of a side-firing optical fiber with capillary and reflective member, according to another embodiment of the invention.

FIG. 4A is a cross-sectional view of a side-firing optical fiber with an optically-opaque capillary and reflective member, according to an embodiment of the invention.

FIG. 4B is a cross-sectional view of a side-firing optical fiber with an optically-opaque capillary and reflective member, according to another embodiment of the invention.

FIG. 5A is a cross-sectional view of a side-firing optical fiber with capillary and reflective member within an outer member, according to an embodiment of the invention.

FIG. 5B is a cross-sectional view of a side-firing optical fiber with capillary and reflective member within an outer member, according to another embodiment of the invention.

FIG. 6A is a cross-sectional view of a side-firing optical fiber with an optically-opaque outer member, according to an embodiment of the invention.

FIG. 6B is a cross-sectional view of a side-firing optical fiber with an optically-opaque outer member, according to another embodiment of the invention.

FIGS. 7A-7F are cross-sectional views of a reflecting member within a capillary, according to embodiments of the invention.

FIGS. 8-10 are flow charts illustrating a method according to an embodiment of the invention.

### DETAILED DESCRIPTION

The devices and methods described herein are generally related to the use of side-firing optical fibers within the body of a patient. For example, the devices and methods can be used in treating symptoms related to an enlarged prostate gland, a condition known as Benign Prostatic Hyperplasia (BPH). BPH is a common condition in which the prostate becomes enlarged with aging. The prostate is a gland that is part of the male reproductive system. The prostate gland includes two lobes that are enclosed by an outer layer of tissue and is located below the bladder and surrounding the urethra, the canal through which urine passes out of the body. Prostate growth can occur in different types of tissue and can affect men differently. As a result of these differences, treatment varies in each case. No cure for BPH exists and once the prostate begins to enlarge, it often continues, unless medical treatment is initiated.

Patients who develop symptoms associated with BPH generally need some form of treatment. When the prostate gland is mildly enlarged, research studies indicate that early treatment may not be needed because the symptoms clear up without treatment in as many as one-third of cases. Instead of immediate treatment, regular checkups are recommended. Only if the condition presents a health risk or the symptoms result in major discomfort or inconvenience to the patient is treatment generally recommended. Current forms of treatment include drug treatment, minimally-invasive therapy, and surgical treatment. Drug treatment is not effective in all cases and a number of procedures have been developed to relieve BPH symptoms that are less invasive than conventional surgery.

While drug treatments and minimally-invasive procedures have proven helpful for some patients, many doctors still recommend surgical removal of the enlarged part of the prostate as the most appropriate long-term solution for patients with BPH. For the majority of cases that require surgery, a procedure known as Transurethral Resection of the Prostate (TURP) is used to relieve BPH symptoms. In this procedure, the medical practitioner inserts an instrument called a resectoscope into and through the urethra to remove the obstructing tissue. The resectoscope also provides irrigating fluids that carry away the removed tissue to the bladder.

More recently, laser-based surgical procedures employing side-firing optical fibers and high-power lasers have been used to remove obstructing prostate tissue. In these procedures, a doctor passes the optical fiber through the urethra using a cystoscope, a specialized endo scope with a small camera on the end, and then delivers multiple bursts of laser energy to destroy some of the enlarged prostate tissue and to shrink the size of the prostate. Patients who undergo laser surgery usually do not require overnight hospitalization and in most cases the catheter is removed the same day or the morning following the procedure. Generally, less bleeding occurs with laser surgery and recovery times tend to be shorter than those of traditional procedures such as TURP surgery.

A common laser-based surgical procedure is Holmium Laser Enucleation of the Prostate (HoLEP). In this procedure, a holmium:YAG (Ho:YAG) laser is used to remove obstructive prostate tissue. The Ho:YAG surgical laser is a solid-state, pulsed laser that emits light at a wavelength of approximately 2100 nm. This wavelength of light is particularly useful for tissue ablation as it is strongly absorbed by water. An advantage of Ho:YAG lasers is that they can be used for both tissue cutting and for coagulation. Another common laser surgery procedure is Holmium Laser Ablation of the Prostate (HoLAP), where a Ho:YAG laser is used to vaporize obstructive prostate tissue. The decision whether to use HoLAP or HoLEP is based primarily on the size of the prostate. For example, ablation may be preferred when the prostate is smaller than 60 cc (cubic centimeters). Laser-based surgical procedures, such as HoLAP and HoLEP, are becoming more preferable because they produce similar results to those obtained from TURP surgery while having fewer complications and requiring shorter hospital stay, shorter catheterization time, and shorter recovery time.

An optical fiber system as described herein can be used to transmit laser energy from a laser source to a target treatment area within a patient's body. The optical fiber system can include a laser source and an optical fiber. One end of the optical fiber can be coupled to the laser source while the other end of the optical fiber, the distal end portion (e.g., the end with a side-firing or laterally-firing portion), can be inserted into the patient's body to provide laser treatment. The distal end portion can include a capillary and a reflective member or reflector within the capillary. An angled or beveled end surface of the reflector disposed within the capillary can redirect laser energy in a lateral direction for side-firing transmission of laser energy to the area of treatment. The angled end surface of the reflector can include, for example, a multilayer dielectric coating. The multilayer dielectric coating can be configured to reflect a portion of the optical beam (e.g., laser beam) from the optical fiber that impinges on the end surface of the reflector at a less glancing angle and would not otherwise be totally internally reflected. In one embodiment, a multilayer dielectric coating can be disposed between a distal end surface of the reflector and an inner portion of the capillary.

It is noted that, as used in this written description and the appended claims, the singular forms “a,” “an” and “the” include plural referents unless the context clearly dictates otherwise. Thus, for example, the term “a wavelength” is intended to mean a single wavelength or a combination of wavelengths. Furthermore, the words “proximal” and “distal” refer to direction closer to and away from, respectively, an operator (e.g., medical practitioner, medical practitioner, nurse, technician, etc.) who would insert the medical device into the patient, with the tip-end (i.e., distal end) of the device inserted inside a patient's body. Thus, for example, the optical fiber end inserted inside a patient's body would be the distal end of the optical fiber, while the optical fiber end outside a patient's body would be the proximal end of the optical fiber.

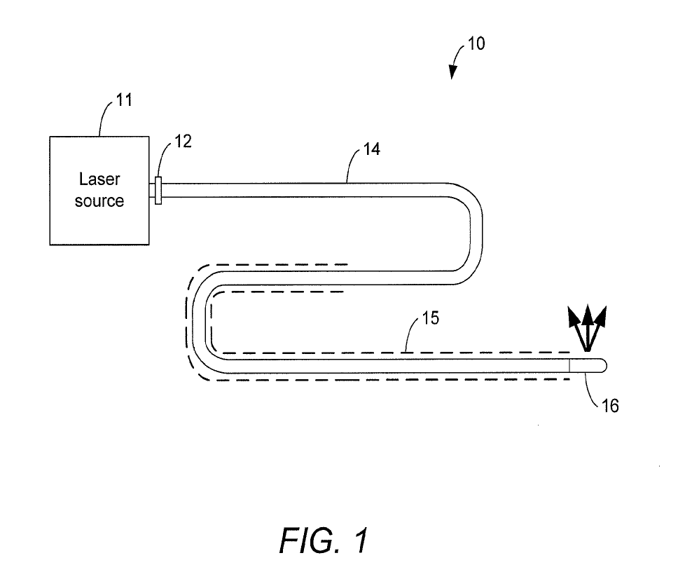

FIG. 1 is a schematic representation of a side-firing optical fiber system according to an embodiment of the invention. An optical fiber side-firing system 10 can include a laser source 11, an optical coupler 12, an optical fiber 14, and an optical-fiber distal end portion 16. The optical fiber side-firing system 10 also includes a suitable catheter or endoscope 15 for inserting the optical-fiber distal end portion 16 into a patient's body. The laser source 11 can include at least one laser that can be used to generate laser energy for surgical procedures. The laser source 11 can include a Ho:YAG laser, for example. The laser source 11 can include at least one of a neodymium-doped:YAG (Nd:YAG) laser, a semiconductor laser diode, or a potassium-titanyl phosphate crystal (KTP) laser, for other examples. In some embodiments, more than one laser can be included in the laser source 11 and more than one laser can be used during a surgical procedure. The laser source 11 can also have a processor that provides timing, wavelength, and/or power control of the laser. For example, the laser source 11 can include mechanisms for laser selection, filtering, temperature compensation, and/or Q-switching operations.

The optical fiber 14 can be coupled to the laser source 11 through the optical coupler 12. The optical coupler 12 can be an SMA connector, for example. The proximal end of the optical fiber 14 can be configured to receive laser energy from the laser source 11, and the distal end of the optical fiber 14 can be configured to output the laser energy through the optical-fiber distal end portion 16. The optical fiber 14 can include, for example, a core, one or more cladding layers about the core, a buffer layer about the cladding, and a jacket. The core can be made of a suitable material for the transmission of laser energy from the laser source 11. In some embodiments, when surgical procedures use wavelengths ranging from about 500 nm to about 2100 nm, the core can be made of silica with a low hydroxyl (OH⁻) ion residual concentration. An example of using low hydroxyl (low-OH) fibers in medical devices is described in U.S. Pat. No. 7,169,140 to Kume, the disclosure of which is incorporated herein by reference in its entirety. The core can be multi-mode and can have a step or graded index profile. The cladding can be a single or a double cladding that can be made of a hard polymer or silica. The buffer can be made of a hard polymer such as Tefzel®, for example. When the optical fiber includes a jacket, the jacket can be made of Tefzel®, for example, or can be made of other polymers.

The endoscope 15 can define one or more lumens. In some embodiments, the endoscope 15 includes a single lumen that can receive therethrough various components such as the optical fiber 14. The endoscope 15 has a proximal end configured to receive the optical-fiber distal end portion 16 and a distal end configured to be inserted into a patient's body for positioning the optical-fiber distal end portion 16 in an appropriate location for a laser-based surgical procedure. For example, to relieve symptoms associated with BPH, the endoscope 15 can be used to place the optical-fiber distal end portion 16 at or near the enlarged portion of the prostate gland. The endoscope 15 includes an elongate portion that can be flexible to allow the elongate portion to be maneuvered within the body. The endoscope 15 can also be configured to receive various medical devices or tools through one or more lumens of the endoscope, such as, for example, irrigation and/or suction devices, forceps, drills, snares, needles, etc. An example of such an endoscope with multiple lumens is described in U.S. Pat. No. 6,296,608 to Daniels et al., the disclosure of which is incorporated herein by reference in its entirety. In some embodiments, a fluid channel (not shown) is defined by the endoscope 15 and coupled at a proximal end to a fluid source (not shown). The fluid channel can be used to irrigate an interior of the patient's body during a laser-based surgical procedure. In some embodiments, an eyepiece (not shown) can be coupled to a proximal end portion of the endoscope 15, for example, and coupled to a proximal end portion of an optical fiber that can be disposed within a lumen of the endoscope 15. Such an embodiment allows a medical practitioner to view the interior of a patient's body through the eyepiece.

The optical-fiber distal end portion 16 can include one or more members, elements, or components that can individually or collectively operate to transmit laser energy in a lateral direction offset from a longitudinal axis or centerline of the distal end of the optical fiber core. In an embodiment, the optical-fiber distal end portion 16 can have a reflector or reflecting member with a multilayer dielectric coating on an angled surface for side-firing laser energy during a surgical procedure. Such a multilayer dielectric coating can be configured to have a high reflectance value (e.g., R>99.9%) at the laser operating wavelength and/or at the desired angle of incidence. In another embodiment, the optical-fiber distal end portion 16 can have a multilayer dielectric coating between a distal end surface of the reflector and an inner portion of the distal end portion of a capillary. In some instances, the optical-fiber distal end portion 16 can include more than one multilayer dielectric coating.

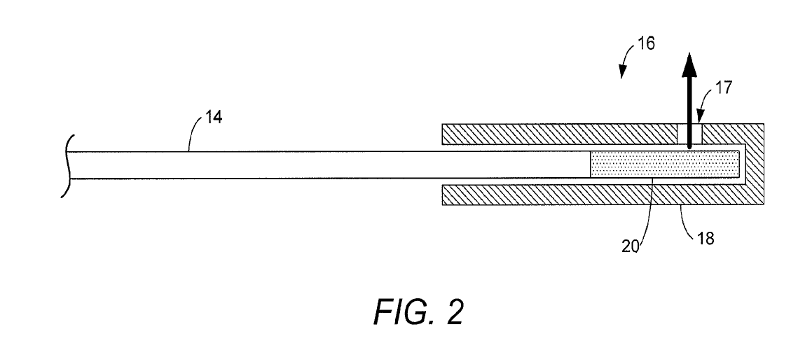

FIG. 2 is a cross-sectional view of an optical-fiber distal end portion according to an embodiment of the invention. The optical-fiber distal end portion 16 can include an inner portion 20 and surrounded by an outer portion 18. The outer portion 18 can include a high-profile member such as, for example, a metal or ceramic cover or cap. The cover or cap is generally made of surgical grade stainless steel or other materials with like properties. In some instances, it can be desirable to have the cap made of a ceramic material (e.g., alumina) because certain ceramics can offer stable characteristics at high-temperatures and/or have a high reflectance value at the laser operating wavelength. The outer portion 18 can provide protection to the optical-fiber distal end portion 16. In some embodiments, the outer portion 18 can include a low-profile cover (e.g., a coating or a sleeve).

The outer portion 18 can include a window or transmissive portion 17 through which laterally-redirected or side-fired laser energy can be transmitted for surgical treatment. For example, when the outer portion 18 is made of an opaque material, a window can be defined after removing at least a portion of the opaque material. In another example, when the outer portion 18 is made of an optically-transmissive material, laser energy can be transmitted or sent through the outer portion 18. In some embodiments, the optically-transmissive material can be treated thermally, optically, mechanically, and/or chemically to improve its structural and/or optical characteristics such that laser energy can be delivered more effectively to the target area. For example, the optically-transmissive material can be thermally treated during manufacturing using a CO₂ laser.

The inner portion 20 can include one or more members, components, and/or devices to redirect laser energy. For example, the inner portion 20 can include a capillary or capillary tube. The capillary can be made of, for example, at least one of silica, sapphire, and/or other like materials. In one embodiment, the inner portion 20 can include a distal end portion of the core of the optical fiber 14 disposed within a capillary. As described below in more detail, the inner portion 20 can also include reflecting members and/or mirrors that can be used to redirect laser energy to provide side-firing operations.

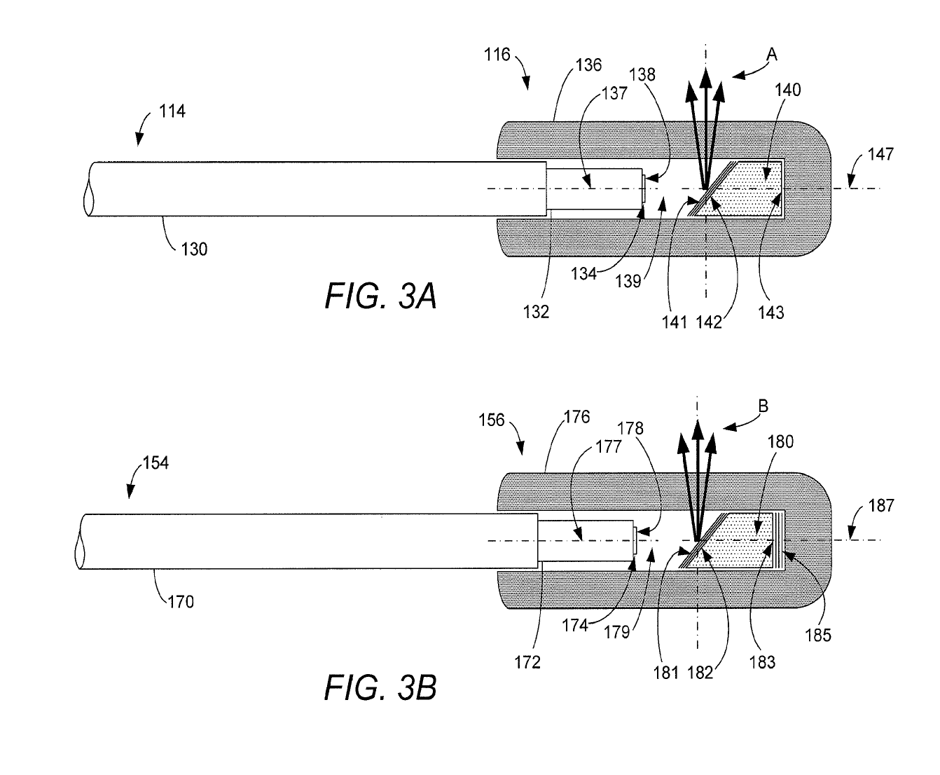

FIG. 3A is cross-sectional view of a side-firing optical fiber with a capillary and a reflective member, according to an embodiment of the invention. The side-firing optical-fiber distal end portion 116 can include a reflective member 140 disposed within a capillary 136. A distal end portion of a buffer layer 130, a distal end portion of a cladding layer 132, and an optical-fiber-core end portion 134 can be disposed within the capillary 136. The optical-fiber-core end portion 134 can include a core-end surface 138 that is substantially perpendicular relative to a longitudinal axis or centerline 137 of the optical-fiber-core end portion 134. In some instances, the distal end of the cladding layer 132 can extend to the distal end of the optical-fiber-core end portion 134 (e.g., the polished end). Laser energy A transmitted through the optical fiber 114 can exit via the core-end surface 138 to be redirected at the reflector 140.

The capillary 136 can be coupled (e.g., affixed)to an outer surface of the distal end portion the buffer layer 130, for example. The overlap between the capillary 136 and the outer surface of the buffer layer 130 can be sufficiently large to provide mechanical stability to the joint or coupling. As shown in FIG. 3A, the capillary 136 can be made of an optically-transmissive material such as, for example, sapphire.

The reflector 140 can include a proximal end surface 142 that is angled relative to a longitudinal axis or centerline 147 of distal end portion of the capillary 136. In some embodiments, the longitudinal axis 147 of the capillary 136 can be substantially parallel to the longitudinal axis 137 of the optical-fiber-core end portion 134. The reflector 140 can also include a distal end surface 143 that is substantially perpendicular relative to the longitudinal axis or centerline 147. The reflector 140 can be made of various materials such as, for example, a polymer, a glass, a metal, and/or a ceramic. The optical, thermal, and/or mechanical properties of a material and/or combination of materials can be considered when determining the appropriate material, shape, and/or size for the reflector 140. For example, substantially matching thermal expansion coefficients for the reflector 140 and the capillary 136 can reduce the effect of mechanical stresses that may occur from overheating in the device. Moreover, material selection may also depend on the manner in which the reflector 140 is to be fixed within the capillary 136. For example, a glass-based reflector 140 and a glass-based capillary 136 can be joined or coupled through a fusion process that uses a CO₂ laser during manufacturing to perform the fusion operation. In this regard, reducing or minimizing the formation of bubbles, air gaps, and/or defects during the fusion process can produce better matching of optical, thermal and/or mechanical properties.

The angled surface 142 can be configured to produce reflection of laser energy that is transmitted through the optical-fiber-core end portion 134 to laterally redirect the laser energy. The angled surface 142 can be used to redirect laser energy in a lateral direction offset from the longitudinal axis or centerline 147 of the distal end portion of the capillary 136. By determining an appropriate angle or configuration for the angled surface 142, the side-fired laser energy A can be transmitted in a lateral direction that is appropriate for laser-based surgical procedures. For example, a 45 degree angle of incidence can result in the laser or optical beam being laterally reflected at an angle of about 90 degrees from the longitudinal axis of the distal end portion of the optical fiber.

The angle of the angled surface 142 can be determined, selected, or designed based on at least one of several parameters. For example, the angle can be configured based on the wavelength of the laser energy A, the exit or output location for the side-fired laser energy A, and/or the optical properties of the capillary 136 and/or the reflector 140. Moreover, the optical properties of a volume or region 139 that remains within the inner portion of the capillary 136 after the disposing of the distal end portion of the optical fiber 114 and the reflector 140 can also be used in determining an appropriate angle for the angled surface 142.

As shown in FIG. 3A, a multilayer dielectric coating 141 can be disposed on the angled surface 142. The multilayer dielectric coating 141 can be used to improve the reflection efficiency of the angled surface 142 over a wider range of angles associated with the laser beam propagation through the optical fiber. The high reflectivity and low optical absorption of multilayer dielectric coatings can reduce the device operating temperature and/or reduce the amount of cooling that may be used to operate the device at a safe temperature.

In some instances, some of the laser energy is not side-fired or laterally-redirected at the angled surface 142 and instead it leaks through the angled surface 142 and is transmitted through the reflector 140. To minimize the effect that this leaked laser energy can have on the operation of the device, a multilayer dielectric coating can be positioned between the distal end of the reflector 140 and an inner side of the distal end of the capillary 136 to reflect the leaked laser energy away from, for example, the distal end portion of the capillary 136.

FIG. 3B is cross-sectional view of a side-firing optical fiber with capillary and reflective member, according to another embodiment of the invention. A shown in FIG. 3B, a multilayer dielectric coating 185 can be disposed between a distal end surface 183 of a reflector 180 and an inner side of a distal end portion of the capillary 176. The distal end surface 183 can be substantially perpendicular to a longitudinal axis or centerline 187 of the distal end portion of the capillary 176. The multilayer dielectric coating 185 can reduce overheating and/or improve efficiency by reflecting laser energy that may have leaked through a multilayer dielectric coating 181 and an angled surface 182 away from the distal end portion of the capillary 176.

Each of the multilayer dielectric coatings shown in FIGS. 3A and 3B can be made of a multiple dielectric layers that collectively and efficiently reflect laser energy. A dielectric layer can be made of alternating layers of SiO₂ (silica) and TiO₂ (titanium dioxide or titania), for example. The multilayer dielectric coatings can include alternating layers of two or more materials each with a different dielectric constant. In some embodiments, the multilayer dielectric coatings can be configured to operate as a ¼ wavelength mirror in which sets of two alternating layers are used and each layer has an optical thickness that is ¼ the wavelength of the laser energy. The multilayer dielectric coatings can be deposited using any of multiple deposition techniques, such as electron beam or ion beam deposition, for example. For instance, the multilayer dielectric coatings 181 and 185 shown in FIG. 3B are different and can be deposited using separate and/or different deposition techniques. Moreover, the multilayer dielectric coatings 181 and 185 can include different number of layers, different number of materials, and/or different layer thicknesses, for example.

FIGS. 3A and 3B each depicts representations of laser energy propagating through an optical path that is at least partly defined by one or more members, elements, and/or components in the capillary. The optical path can include multiple segments through which the laser energy propagates to provide side-fired laser-based medical treatment. For example, as shown in FIG. 3A, a first portion or segment of the optical path can be defined by the core-end surface 138 and the reflector 140. A second portion or segment of the optical path can be defined by, for example, the reflector 140 and the portion of the capillary 136 through which the laser energy A is transmitted. In this regard, the reflector 140 can be within the optical path and define one or more of the segments of the optical path. The second segment of the optical path can be non-parallel to the first segment of the optical path.

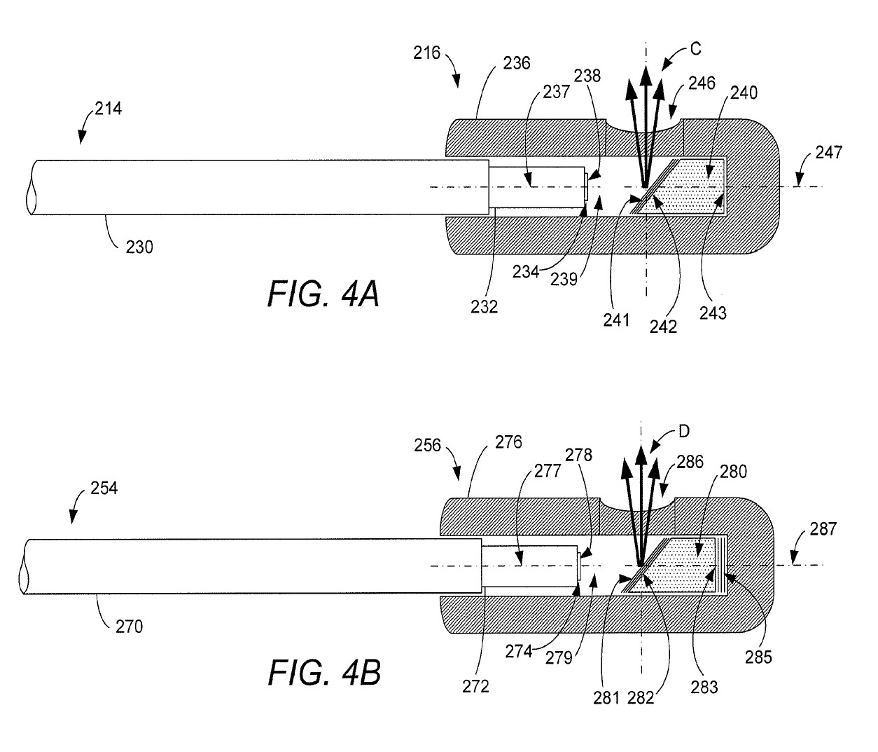

FIG. 4A is a cross-sectional view of a side-firing optical fiber with an optically-opaque capillary and reflective member according to an embodiment of the invention. The side-firing optical-fiber distal end portion 216 can include a reflective member 240 and an optical-fiber-core end portion 234 disposed within a capillary 236. The optical-fiber-core end portion 234 can include a core-end surface 238 that is substantially perpendicular to a longitudinal axis or centerline 237 of the optical-fiber-core end portion 234. Laser energy C transmitted through the optical fiber 214 can exit via the core-end surface 238 to be redirected at the reflector 240.

The capillary 236 can be coupled (e.g., affixed) to an outer surface of the distal end portion of the buffer layer 230, for example. The overlap between the capillary 236 and the outer surface of the buffer layer 230 can be sufficiently large to provide mechanical stability to the joint or coupling. As shown in FIG. 4A, the capillary 236 can be made of an optically-opaque material. In this regard, a window or transmissive portion 246 may be defined through which the laser energy C can be transmitted during a surgical procedure. The window 246 can be offset from a longitudinal axis or centerline 247 of the distal end portion of the capillary 236. The optical-fiber-core end portion 234, the reflector 240, and/or a multilayer dielectric coating 241 on an angled surface 242 of the reflector 240 can be collectively configured to redirect the laser energy C transmitted from the core-end surface 238 in a side-fired direction and through the window 246.

FIG. 4B is a cross-sectional view of a side-firing optical fiber with an optically-opaque capillary and reflective member according to another embodiment of the invention. As shown in FIG. 4B, a multilayer dielectric coating 285 can be disposed between a distal end surface 283 of a reflector 280 and an side of a distal end portion of a capillary 276. The distal end surface 283 can be substantially perpendicular to a longitudinal axis or centerline 287 of the distal end portion of the capillary 276. The multilayer dielectric coating 285 can reduce overheating and/or improve efficiency by reflecting laser energy that may have leaked through a multilayer dielectric coating 281 and through an angled surface 282 away from the capillary 276.

As described above, each of the multilayer dielectric coatings shown in FIGS. 4A and 4B can be made of multiple dielectric layers that collectively operate to reflect laser energy. The multilayer dielectric coatings 281 and 285 shown in FIG. 4B are different and can be deposited using separate and/or different deposition techniques. Moreover, the multilayer dielectric coatings 281 and 285 can include different number of layers, different number of materials, and/or different layer thicknesses, for example.

FIGS. 4A and 4B each depicts representations of laser energy propagating through an optical path that is at least partly defined by one or more members, elements, and/or components in the capillary. The optical path can include multiple segments through which the laser energy propagates to provide side-fired laser-based medical treatment. For example, as shown in FIG. 4A, a first portion or segment of the optical path can be defined by the core-end surface 238 and the reflector 240. A second portion or segment of the optical path can be defined by, for example, the reflector 240 and the window 246 through which the laser energy C is transmitted. In this regard, the reflector 240 can be within the optical path and define one or more of the segments of the optical path. Similarly, the window 246 can be within the optical path and define one or more of the segments of the optical path. The second segment of the optical path can be non-parallel to the first segment of the optical path.

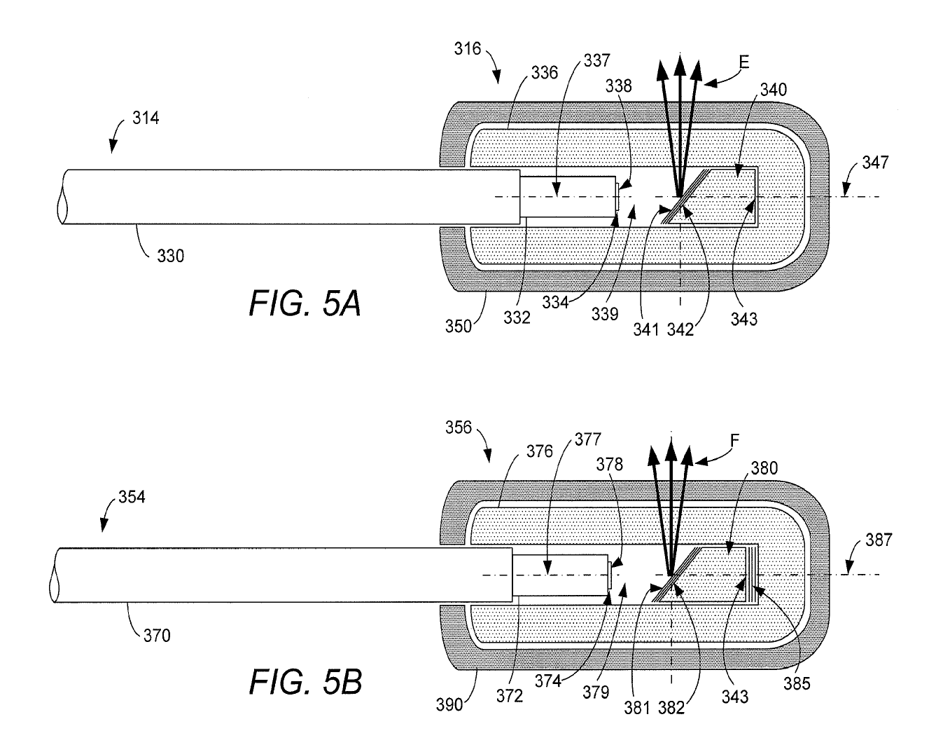

FIG. 5A is a cross-sectional view of a side-firing optical fiber with capillary and reflective member within an outer member according to an embodiment of the invention. The side-firing optical-fiber distal end portion 316 can include a capillary 336 disposed within an outer member 350. In some embodiments, the outer member 350 can be a high-profile member such as, for example, a metal cap. In other embodiments, the outer member 350 can be a low-profile member such as, for example, a polymer-based coating or sleeve. A reflective member 340 and an optical-fiber-core end portion 334 can be disposed within the capillary 336. The optical-fiber-core end portion 334 can include a core-end surface 338 that is substantially perpendicular to a longitudinal axis or centerline 337 of the optical-fiber-core end portion 334. Laser energy E transmitted through the optical fiber 314 can exit via the core-end surface 338 to be redirected at the reflector 340. In this regard, the optical-fiber-core end portion 334, the reflector 340, and/or a multilayer dielectric coating 341 on an angled surface 342 of the reflector 340 can be collectively configured to redirect the laser energy E transmitted from the core-end surface 338 in a side-fired direction that passes through the capillary 336 and through the outer member 350.

The capillary 336 can be coupled (e.g., affixed) to a portion of an outer surface of the distal end portion the buffer layer 330, for example. The overlap between the capillary 336 and the outer surface of the buffer layer 330 can be sufficiently large to provide mechanical stability to the joint or coupling. Similarly, the outer member 350 can be coupled to another portion of the outer surface of the distal end portion the buffer layer 330, for example. In some embodiments, the overlap between the outer member 350 and the outer surface of the buffer layer 330 can be sufficiently large to provide mechanical stability to the joint or coupling. As shown in FIG. 5A, the capillary 336 and the outer member 350 can both be made of an optically-transmissive material through which the laser energy E can be side-fired during laser-based surgical procedures.

FIG. 5B is a cross-sectional view of a side-firing optical fiber with capillary and reflective member within an outer member according to another embodiment of the invention. A shown in FIG. 5B, a multilayer dielectric coating 385 can be disposed between a distal end surface 383 of a reflector 380 and an side of a distal end portion of a capillary 376. The distal end surface 383 can be substantially perpendicular to a longitudinal axis or centerline 387 of the distal end portion of the capillary 376. The multilayer dielectric coating 385 can reduce overheating and/or improve efficiency by reflecting laser energy that may have leaked through a multilayer dielectric coating 381 and through an angled surface 382 away from the capillary 376.

As described above, each of the multilayer dielectric coatings shown in FIGS. 5A and 5B can be made of multiple dielectric layers that collectively operate to reflect laser energy. The multilayer dielectric coatings 381 and 385 shown in FIG. 5B are different and can be deposited using separate and/or different deposition techniques. Moreover, the multilayer dielectric coatings 381 and 385 can include different number of layers, different number of materials, and/or different layer thicknesses, for example.

FIGS. 5A and 5B each depicts representations of laser energy propagating through an optical path that is at least partly defined by one or more members, elements, and/or components in the capillary and/or the outer member. The optical path can include multiple segments through which the laser energy propagates to provide side-fired laser-based medical treatment. For example, as shown in FIG. 5A, a first portion or segment of the optical path can be defined by the core-end surface 338 and the reflector 340. A second portion or segment of the optical path can be defined by, for example, the reflector 340 and the portion of the capillary 336 through which the laser energy E is transmitted. In another example, the second segment of the optical path can be defined by the reflector 340 and the portion of the outer member 350 through which the laser energy E is transmitted. In yet another example, a segment of the optical path can be defined by the portions of the capillary 336 and the outer member 350 through which the laser energy E is transmitted. In the above described examples, the reflector 340, the capillary 336, and/or the outer member 350 can be within the optical path and can define one or more of the segments of the optical path. The segments of the optical path described above need not be parallel to each other.

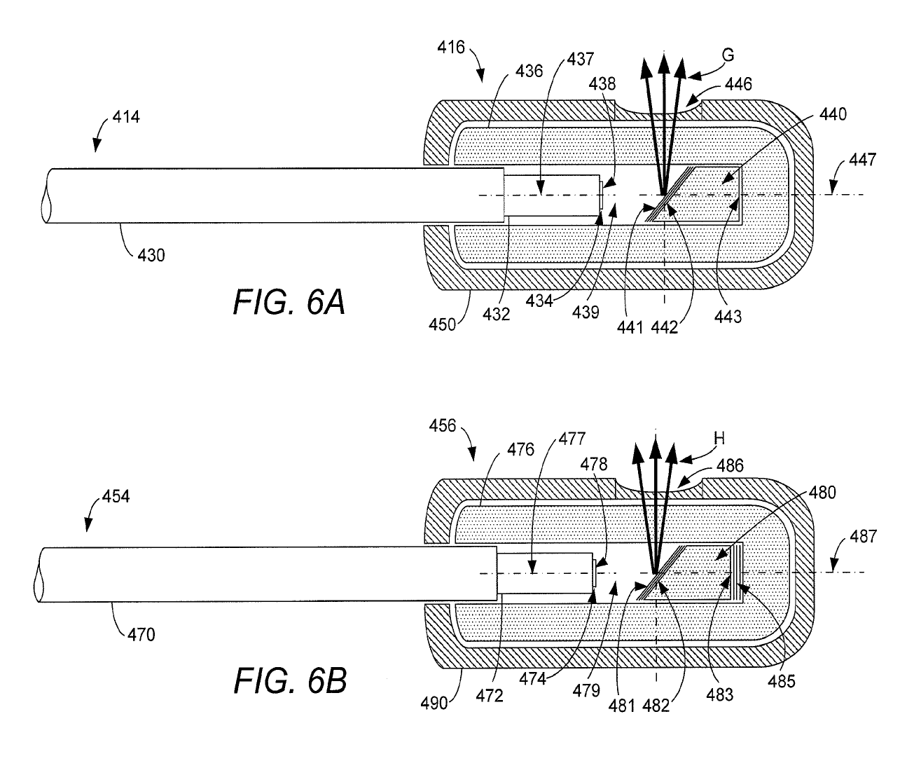

FIG. 6A is a cross-sectional view of a side-firing optical fiber with an optically-opaque outer member, according to an embodiment of the invention. The side-firing optical-fiber distal end portion 416 can include a capillary 436 disposed within an outer member 450. An optical-fiber-core end portion 434 and a reflective member 440 can be disposed within the capillary 436. The optical-fiber-core end portion 434 can include a core-end surface 438 that is substantially perpendicular to a longitudinal axis or centerline 437 of the optical-fiber-core end portion 434. Laser energy G transmitted through the optical fiber 414 can exit via the core-end surface 438 to be redirected at the reflector 440 in a side-fired direction that passes through the capillary 436 and through the outer member 450.

As shown in FIG. 6A, the capillary 436 can be made of an optically-transmissive material and the outer member 450 can be made of an optically-opaque material. In this regard, a window or transmissive portion 446 on the outer member 450 may be defined through which the laser energy G can be transmitted during a surgical procedure. The window 446 can be offset from a longitudinal axis or centerline 447 of the distal end portion of the capillary 436. The optical-fiber-core end portion 434, the reflector 440, and/or a multilayer dielectric coating 441 on an angled surface 442 of the reflector 440 can be collectively configured to redirect the laser energy G transmitted from the core-end surface 438 in a side-fired direction that passes through the capillary 436 and through the window 446.

FIG. 6B is a cross-sectional view of a side-firing optical fiber with an optically-opaque outer member, according to another embodiment of the invention. As shown in FIG. 6B, a multilayer dielectric coating 485 can be disposed between a distal end surface 483 of a reflector 480 and an inner side of a distal end portion of a capillary 476. The distal end surface 483 can be substantially perpendicular to a longitudinal axis or centerline 487 of the distal end portion of the capillary 476. The multilayer dielectric coating 485 can reduce overheating and/or improve efficiency by reflecting laser energy that may have leaked through a multilayer dielectric coating 481 and through an angled surface 482 away from the capillary 476.

As described above, each of the multilayer dielectric coatings shown in FIGS. 6A and 6B can be made of multiple dielectric layers that collectively operate to reflect laser energy. The multilayer dielectric coatings 481 and 485 shown in FIG. 6B are different and can be deposited using separate and/or different deposition techniques. Moreover, the multilayer dielectric coatings 481 and 485 can include different number of layers, different number of materials, and/or different layer thicknesses, for example.

FIGS. 6A and 6B each depicts representations of laser energy propagating through an optical path that is at least partly defined by one or more members, elements, and/or components in the capillary and/or the outer member. The optical path can include multiple segments through which the laser energy propagates to provide side-fired laser-based medical treatment. For example, as shown in FIG. 6A, a first portion or segment of the optical path can be defined by the core-end surface 438 and the reflector 440. A second portion or segment of the optical path can be defined by, for example, the reflector 440 and the portion of the capillary 436 through which the laser energy G is transmitted. In another example, the second segment of the optical path can be defined by the reflector 440 and the window 446 through which the laser energy G is transmitted. In yet another example, a segment of the optical path can be defined by the portion of the capillary 436 through which the laser energy E is transmitted and the window 446. In the above described examples, the reflector 440, the capillary 436, and/or the window 446 can be within the optical path and can define one or more of the segments of the optical path.

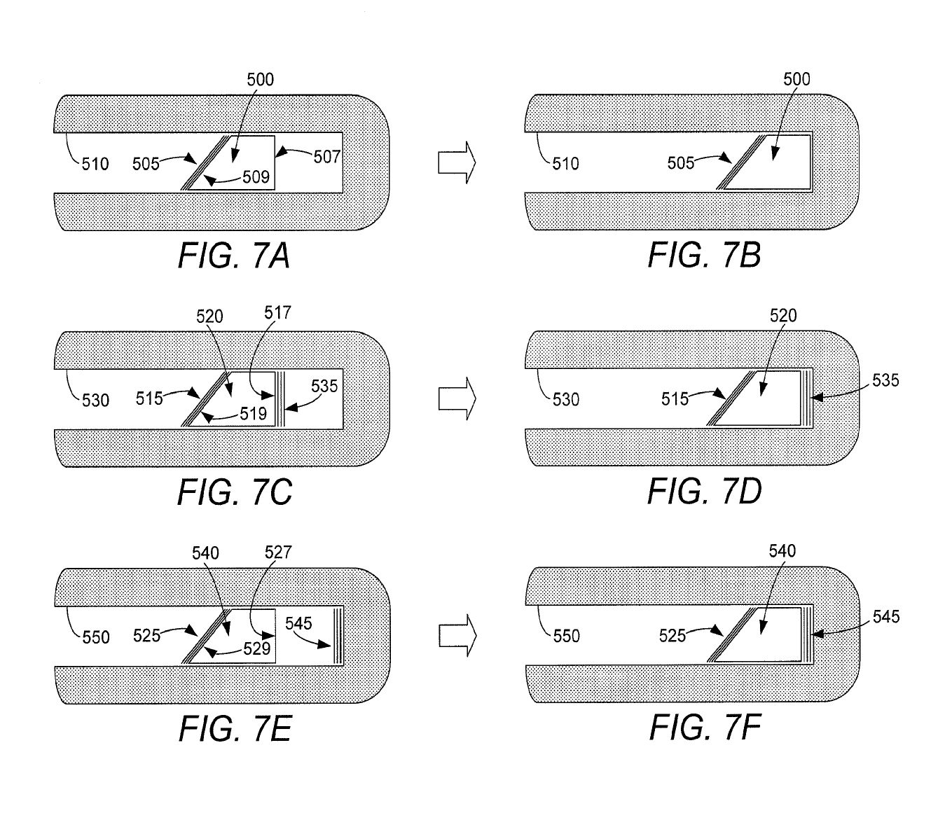

FIGS. 7A-7F are cross-sectional views of a reflecting member within another member, according to embodiments of the invention. As shown in FIGS. 7A and 7B, a reflecting member 500 can be disposed within an inner portion of a member 510. The member 510 can be a capillary or capillary tube, for example. A multilayer dielectric coating 505 can be disposed on an angled surface 509 of the reflecting member 500. A distal end surface 507 of the reflecting member 500 can be positioned proximate to an inner side of the distal end portion of the member 510.

As shown in FIGS. 7C and 7D, a reflecting member 520 can be disposed within an inner portion of a member 530. The member 530 can be a capillary or capillary tube, for example. A first multilayer dielectric coating 515 can be disposed on an angled surface 519 of the reflecting member 520. A second multilayer dielectric coating 535 can be disposed on a distal end surface 517 of the reflecting member 520. The distal end surface 517 can be positioned proximate to an inner side of a distal end portion of the member 530 such that the second multilayer dielectric coating 535 is positioned between the reflecting member 520 and the inner side of the distal end portion of the member 530.

As shown in FIGS. 7E and 7F, a reflecting member 540 can be disposed within an inner portion of a member 550. The member 550 can be a capillary or capillary tube, for example. A first multilayer dielectric coating 525 can be disposed on an angled surface 529 of the reflecting member 540. A second multilayer dielectric coating 545 can be disposed on an inner side of a distal end portion of the member 550. A distal end surface 527 of the reflecting member 540 can be positioned proximate to the inner side of the distal end portion of the member 550 such that the second multilayer dielectric coating 545 is positioned between the reflecting member 540 and inner side of the distal end portion of the member 550.

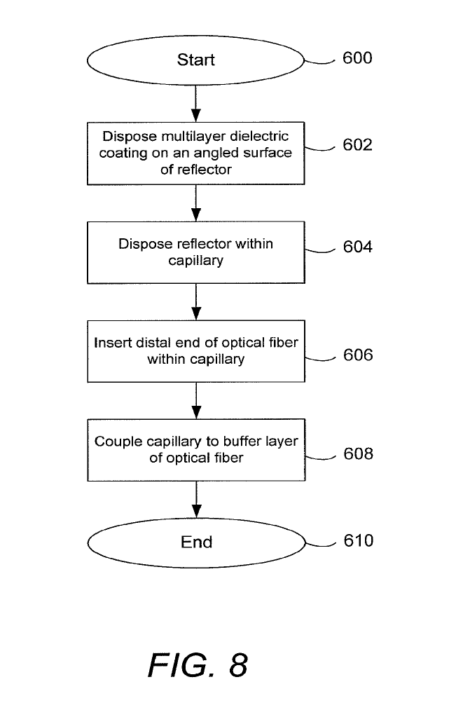

FIG. 8 is a flow chart illustrating a method for manufacturing a side-firing optical fiber, according to an embodiment of the invention. At 602, after start 600, a multilayer dielectric coating is disposed on a proximal end surface of a reflector. The proximal end surface of the reflector can be angled relative to a longitudinal axis or centerline of a distal end portion of a capillary. At 604, the coated reflector can be disposed within an inner region of the capillary. In some embodiments, the capillary can be optically-transmissive. In other embodiments, the capillary can be optically-opaque and include an opening or window through which laser energy can be transmitted. In some embodiments, the position of the coated reflector can be fixed by, for example, a fusion process that fuses at least a portion of the reflector to the capillary. At 606, a distal end portion of an optical fiber can be disposed within the capillary. A distal end of the optical fiber core and the coated reflector can be collectively configured to redirect laser energy in a side-fired direction that is offset from the longitudinal axis of the distal end portion of the capillary. At 608, the proximal end portion of the capillary can be coupled (e.g., affixed) to an outer surface of a buffer layer of the optical fiber. The overlap between the capillary and the outer surface of the buffer layer can be sufficiently large to provide mechanical stability to the coupling. In some embodiments, the capillary can be disposed within an outer member such as, a polymer-based coating or a metal or ceramic cap, for example. The outer member can be optically-transmissive or optically-opaque and include an opening or window through which laser energy can be transmitted. After 608, the method can proceed to end 610.

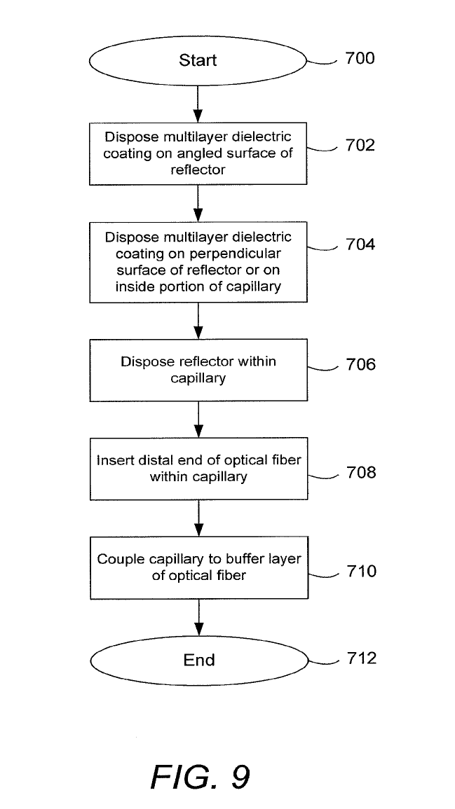

FIG. 9 is a flow chart illustrating a method for manufacturing a side-firing optical fiber, according to another embodiment of the invention. At 702, after start 700, a first multilayer dielectric coating can be disposed on a proximal end surface of a reflector. The proximal end surface of the reflector can be angled relative to a longitudinal axis or centerline of a distal end portion of a capillary. At 704, a second multilayer dielectric coating can be disposed on a distal end surface of the reflector. The distal end surface of the reflector can be substantially perpendicular to the longitudinal axis or centerline of the distal end portion of the capillary. Optionally, the second multilayer dielectric coating can be disposed on a distal end portion of the inner portion of the capillary. The first and second multilayer dielectric coatings can be different and can be deposited using separate and/or different deposition techniques. Moreover, the first and second multilayer dielectric coatings can include different number of layers, different number of materials, and/or different layer thicknesses, for example.

At 706, the coated reflector can be disposed within an inner region of the capillary. In some embodiments, the capillary can be optically-transmissive. In other embodiments, the capillary can be optically-opaque and include an opening or window through which laser energy can be transmitted. In some embodiments, the position of the coated reflector can be fixed by, for example, a fusion process that fuses at least a portion of the reflector to the capillary. At 708, a distal end portion of an optical fiber can be disposed within the capillary. A distal end of the optical fiber core and the coated reflector can be collectively configured to redirect laser energy in a side-fired direction that is offset from the longitudinal axis of the distal end portion of the capillary. At 710, the proximal end portion of the capillary can be coupled (e.g., affixed) to an outer surface of a buffer layer of the optical fiber. The overlap between the capillary and the outer surface of the buffer layer can be sufficiently large to provide mechanical stability to the coupling. In some embodiments, the capillary can be disposed within an outer member such as, a polymer-based coating or a metal or ceramic cap, for example. The outer member can be optically-transmissive or optically-opaque and include an opening or window through which laser energy can be transmitted. After 710, the method can proceed to end 712.

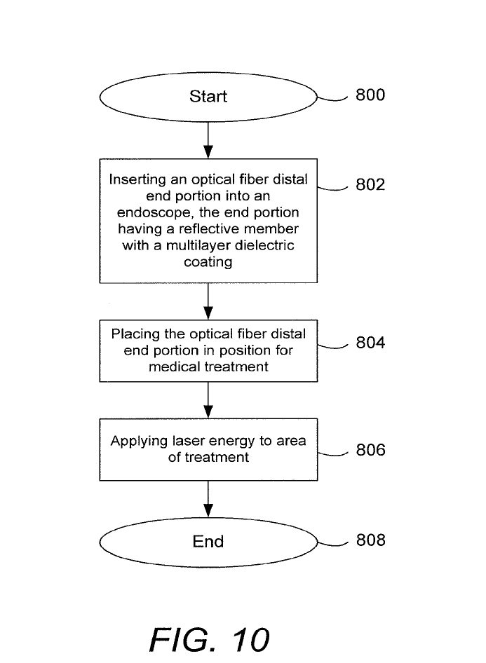

FIG. 10 is a flow chart illustrating a method of using an optical fiber side-firing system, according to another embodiment of the invention. At 802, after start 800, an optical-fiber distal end portion can be inserted within an inner portion or lumen of an endoscope. The optical-fiber distal end portion includes a reflector inside a capillary and a multilayer dielectric coating on an angled surface of the reflector. The optical-fiber distal end portion can include a second multilayer dielectric coating positioned between the reflector and a distal end of the inner portion of the capillary. At 804, the endoscope can be at least partially inserted into the patient's body during a laser-based surgical procedure. Once inserted into the patient's body, the endoscope can be used to place or position the optical-fiber distal end portion at or near the area of treatment. At 806, laser energy from a laser source can be transmitted through the optical fiber such that laser energy is side-fired or laterally redirected to the treatment area. After 806, the method can proceed to end 808.

### CONCLUSION

While various embodiments have been described above, it should be understood that they have been presented by way of example only, and not limitation. For example, the optical fiber side-firing system described herein can include various combinations and/or sub-combinations of the components and/or features of the different embodiments described. Although described with reference to use for treatment of symptoms related to BPH, it should be understood that the optical fiber side-firing system and the side-firing optical fibers, as well as the methods of using the optical fiber side-firing system and the side-firing optical fibers can be used in the treatment of other conditions.

Embodiments of a side-firing optical fiber can also be provided without the optical fiber side-firing system described herein. For example, a side-firing optical fiber can be configured to be used with other laser sources, endoscopes, etc., not specifically described herein. A side-firing optical fiber can have a variety of different shapes and sizes than as illustrated and described herein. A side-firing optical fiber can also include other features and/or components such as, for example, lenses and/or filters.

In one embodiment, an apparatus can include a member having a distal end portion configured to be inserted into a patient's body. The apparatus can include a reflector and a multilayer dielectric coating. The reflector can be disposed within the member. The reflector can be fused to the member, for example. The reflector can have a proximal end portion that includes a surface and the surface can be angled relative to a longitudinal axis of the distal end portion of the member. The angled surface can be configured to redirect laser energy transmitted from a distal end portion of an optical fiber to a lateral direction offset from the longitudinal axis. The member can be disposed within an outer member. The outer member can include a polymer-based coating, a metal cap, and/or a ceramic cap, for example.

The multilayer dielectric coating can be disposed on the angled surface. A multilayer dielectric coating can be disposed on a distal end surface of the reflector. The distal end surface of the reflector can be substantially perpendicular relative to the longitudinal axis. The multilayer dielectric coating can include multiple layers having a first set of layers with an index of refraction and a second set of layers with an index of refraction different than the index of refraction of the first set of layers. The multiple layers of the multilayer dielectric coating can be alternating layers from the first set of layers and the second set of layers. In some instances, a multilayer dielectric coating can disposed on an inner portion of the distal end portion of the member.

The member can include a transmissive portion. The member can include a window offset from a centerline defined by the distal end portion of the member. The member can be made from a ceramic, a sapphire, and/or a stainless steel, for example.

In another embodiment, an apparatus can include a first member, a second member, and a multilayer dielectric coating. The first member can have a distal end portion configured to be inserted into a patient's body. The second member can be disposed within the first member. The second member can have a surface configured to redirect laser energy from a first portion of an optical path to a second portion of the optical path. The second portion of the optical path can be non-parallel to the first portion of the optical path.

The first portion of the optical path can be defined by a distal end portion of an optical fiber. The second portion of the optical path can be offset from a centerline of the distal end portion of the first member. In some instances, the optical path can include a multiple segments.

The multilayer dielectric coating can be disposed on the surface of the second member. The surface of the second member can be angled relative to a centerline of the distal end portion of the first member. A multilayer dielectric coating can be disposed on a distal end surface of the second member that is substantially perpendicular relative to a longitudinal axis of a distal end portion of the first member. A multilayer dielectric coating can be disposed on an inner portion of the first member.

In some instances, the first member can be disposed within a third member. The third member can include a polymer-based coating, a metal cap, and/or a ceramic cap, for example.

In another embodiment, a method can include disposing a multilayer dielectric coating on a surface of a reflector. The surface of the reflector can be angled relative to a longitudinal axis of a distal end portion of a member. The process can also include disposing the reflector within the member and disposing a distal end portion of an optical fiber within the member. The distal end portion of the optical fiber and the angled surface can be collectively configured to laterally redirect laser energy.

Moreover, the method can include disposing a multilayer dielectric coating on a distal end portion of the reflector. The distal end portion of the reflector having a substantially perpendicular surface relative to the longitudinal axis of the distal end portion of the member. A multilayer dielectric coating can be disposed on an inner portion of the distal end portion of the member.

In some instances, the method can include disposing a window in the distal end portion of the member. The distal end portion of the member defines a centerline and the window is offset from the centerline. The method can also include fixedly coupling the reflector to the inner portion of the distal end portion of the member and/or fixedly coupling the distal end portion of the optical fiber to the member. The method can include disposing the member within an outer member.

In another embodiment, a method can include disposing a multilayer dielectric coating on a surface of a first member and disposing the first member within an optical path and inside a second member. The surface of the first member can be angled relative to a longitudinal axis of a distal end portion of the second member. The method can also include coupling a distal end portion of an optical fiber with a proximal end of the optical path. The distal end portion of the optical fiber and the angled surface can be collectively configured to redirect laser energy to a distal end of the optical path. The distal end of the optical path can be offset from a centerline defined by a longitudinal axis of the distal end portion of the second member.

Moreover, the method can include disposing a multilayer dielectric coating on an inner portion of the distal end portion of the second member and/or disposing a multilayer dielectric coating on a distal surface of the first member substantially perpendicular relative to the longitudinal axis.

In some instances, the method can include disposing the second member within a third member and/or fixedly coupling the first member to an inner portion of the distal end portion of the second member.

In another embodiment, a method can include inserting a distal end portion of a first member into a patient's body. The first member having a reflector disposed within the first member. The first member having a surface configured to redirect laser energy from a first portion of an optical path to a second portion of the optical path. The second portion of the optical path can be non-parallel to the first portion of the optical path. A multilayer dielectric coating can be disposed on the surface of the reflector. The method can include, after the inserting, activating a laser source to transmit laser energy to the patient's body such that the transmitted laser energy passes through the optical path. The reflector can be disposed within a second member. The second member can be disposed within the first member.

What is claimed is:

1. An apparatus, comprising:
a member having a distal end portion configured to be inserted into a patient's body;
a reflector disposed within the member, the reflector having a proximal end portion including a surface angled relative to a longitudinal axis of the distal end portion of the member, the angled surface configured to redirect laser energy transmitted from a distal end portion of an optical fiber to a lateral direction offset from the longitudinal axis; and
a multilayer dielectric coating disposed on the angled surface.

2. The apparatus of claim 1, further comprising a multilayer dielectric coating disposed on a distal end surface of the reflector substantially perpendicular relative to the longitudinal axis.

3. The apparatus of claim 1, further comprising a multilayer dielectric coating disposed on an inner portion of the distal end portion of the member.

4. The apparatus of claim 1, wherein the multilayer dielectric coating includes a plurality of layers having a first set of layers having an index of refraction and a second set of layers having an index of refraction different than the index of refraction of the first set of layers, the plurality of layers alternating layers from the first set of layers and the second set of layers.

5. The apparatus of claim 1, wherein the member includes a transmissive portion.

6. The apparatus of claim 1, wherein the member includes a window, the distal end portion of the member defining a centerline, the window being offset from the centerline.

7. The apparatus of claim 1, wherein the member is made from at least one of a ceramic, a sapphire, or a stainless steel.

8. The apparatus of claim 1, further comprising an outer member, the member being disposed within the outer member.

9. The apparatus of claim 1, wherein the reflector is fused to the member.

10. A method, comprising:
disposing a multilayer dielectric coating on a surface of a reflector, the surface of the reflector being angled relative to a longitudinal axis of a distal end portion of a member;
disposing the reflector within the member; and
disposing a distal end portion of an optical fiber within the member, the distal end portion of the optical fiber and the angled surface collectively configured to laterally redirect laser energy.

11. The method of claim 10, further comprising disposing a multilayer dielectric coating on a distal end portion of the reflector, the distal end portion of the reflector having a substantially perpendicular surface relative to the longitudinal axis of the distal end portion of the member.

12. The method of claim 10, further comprising disposing a multilayer dielectric coating on an inner portion of the distal end portion of the member.

13. The method of claim 10, further comprising disposing a window in the distal end portion of the member, the distal end portion of the member defining a centerline, the window being offset from the centerline.

14. The method of claim 10, further comprising fixedly coupling the reflector to an inner portion of the distal end portion of the member.

15. The method of claim 10, further comprising fixedly coupling the distal end portion of the optical fiber to the member.

16. The method of claim 10, further comprising disposing the member within an outer member.

17. The method of claim 10, wherein the member is first member, the method further comprising:
disposing the first member within a second member.

18. A method, comprising:
inserting a distal end portion of a first member into a patient's body, a reflector disposed within the first member having a surface configured to redirect laser energy from a first portion of an optical path to a second portion of the optical path non-parallel to the first portion of the optical path, a multilayer dielectric coating being disposed on the surface of the reflector; and
after the inserting, activating a laser source to transmit laser energy to the patient's body, the transmitted laser energy passing through the optical path.

19. The method of claim 18, wherein the reflector is disposed within a second member, the second member being disposed within the first member.

20. The method of claim 18, wherein the multilayer dielectric is coated on a distal surface of the first member substantially perpendicular relative to a longitudinal axis of the distal end portion.
